(19) United States
(12) Patent Application Publication
(10) Pub. No.: US 2013/0178436 A1
(43) Pub. Date: Jul. 11, 2013

(54) COMPOSITION FOR PREVENTING, IMPROVING, OR TREATING RENAL DISEASE INCLUDING MAILLARD BROWNING REACTION PRODUCTS OF PANAX SPECIES PLANT EXTRACT

(71) Applicant: Korea Institute of Science and Technology, Seoul (KR)

(72) Inventors: Ki Sung Kang, Daejeon (KR); Su-Nam Kim, I, Dangneung-si (KR); Jungyeob Ham, Seoul (KR); Woojung Lee, Seongnam-si (KR); Noriko Yamabe, Gangneung-si (KR); Ji Hwan Lee, Gangneung-si (KR)

(73) Assignee: Korea Institute of Science and Technology, Seoul (KR)

(21) Appl. No.: 13/737,482

(22) Filed: Jan. 9, 2013

(30) Foreign Application Priority Data
Jan. 10, 2012 (KR) 10-2012-0003091
Mar. 28, 2012 (KR) 10-2012-0031828

(51) Int. Cl. A61K 31/704

(52) U.S. Cl. CPC A61K 31/704
USPC 514/34

(57) ABSTRACT

A composition for preventing, improving, or treating renal diseases, the composition including as an active ingredient a Maillard browning reaction product obtained by reacting ginsenoside Re, an extract of Panax species plant including ginsenoside Re, or glucose with amino acid at a temperature of 100 to 130° C. for 0.5 to 12 hours.

### CROSS-REFERENCE TO RELATED PATENT APPLICATION

This application claims the benefit of Korean Patent Application No. 10-2012-0003091, filed on Jan. 10, 2012, and Korean Patent Application No. 10-2012-0031828, filed on Mar. 28, 2012, in the Korean Intellectual Property Office, the disclosures of which are incorporated herein in their entirety by reference.

### BACKGROUND OF THE INVENTION

1. Field of the Invention

One or more embodiments of the present invention relate to a composition for preventing, improving, or treating a renal disease, and more particularly, to a composition for preventing, improving, or treating a renal disease which includes as an active ingredient Maillard browning reaction products of an extract of Panax species plant.

2. Description of the Related Art

The kidneys are excretory organs which play a vital role in excretion of waste through urine in the human body. A variety of medicaments or environmental pollutants exhibit toxicity against the kidneys, and representative drugs that induce nephrotoxicity include non-steroidal anti-inflammatory drugs such as aspirin, indomethacin, and the like; anticancer agents such as puromycin, daunomycin, cyclophosphamide, penicillamine, adriamycin, cisplatin, and the like; immunosuppressive agents; aminoglycoside-based antibiotic agents such as amikacin, gentamicin, kanamycin, neomycin, sisomycin, streptomycin, tobramycin, and the like; cephalosporin-based antibiotic agents; carbapenem-based antibiotic agents such as imiphenem, melophenem, and the like; heavy metals such as cadmium, lead, mercury, chromium, and the like; inorganic and organic heavy metal compounds; compounds such as chloroform, D-serine, sulfonamide, 2-bromoethylene, hydrobromide, and the like; and mycotoxins such as ochratoxin and citrinin. Nephrotoxicity caused by these various medicaments or environmental pollutants is expressed mainly by oxidative stress due to malfunction of the antioxidant defense system in the human body as well as oxidative damage by free radicals. Examples of renal diseases caused by the oxidative stress include, but are not limited to, nephritis, pyelitis, nephrotic syndrome, renal cancer, acute pyelonephritis, chronic pyelonephritis, renal tuberculosis, urinary tract infection, ureterolithiasis, ureteral stone, acute renal failure, chronic renal failure, diabetic nephropathy, chronic glomerulonephritis, acute progressive nephritis, nephrotic syndrome, focal segmental glomerulosclerosis, membranous glomerulonephritis, or membranoproliferative glomerulonephritis.

Therefore, a variety of synthetic antioxidants for reducing oxidative damage due to generation of free radicals and improving oxidative stress by improving the antioxidant activating system in a living body in order to prevent or treat renal diseases have been proposed. Due to problems regarding stability, however, an importance of antioxidants derived from foods and natural substances has been underscored. Thus, various studies using natural substance-derived antioxidants having a free radical scavenging ability have been done (see Ramkumar et al., Food Chem. Toxicol., 47(10), pp. 2516-2521, 2009 and Kumarappan et al., Ren. Fail., 30(3), pp. 307-322, 2008). However, there is still a need to develop a natural substance-derived antioxidant for effectively preventing or treating nephrotoxicity caused by these nephrotoxicity-inducing materials.

Panax ginseng is a perennial plant belonging to the Panax species, Araliaceae family. Examples of Panax species plants having a similar efficacy to Panax ginseng include Panax quinquefolia, Panax notoginseng, Panax japonica, Panax Trifolia, Panax pseudoginseng, Panax vietnamensis, and the like. These Panax species plants contain dammarane-based saponin in common with 1 to 4 saccharide(s) combined to a dammarane backbone, unlike other plants. In particular, saponins contained at high concentration in ginseng, include ginsenosides Rb1, Rb2, Rc, Rd, Rg1, and Re. These saponins have a variety of pharmaceutical effects that greatly differ in types and intensities depending on the structures thereof. The dammarane-based saponin of the Panax species plants has protopanaxadiol or protopanaxatriol as a mother nucleus, and may be classified as illustrated in FIG. 1.

Research into the development of a method for increasing a pharmaceutical effect of ginseng by conversion of the dammarane-based saponin by high temperature, high pressure thermal processing has been conducted. As a representative example, Park et al. developed a processed ginseng extract having an increased ratio of ginsenosides Rg3 and Rg5 to ginsenosides Rb1 and Rb2 by heat-treating ginseng at a high temperature (see U.S. Patent No.: 5,777,460). Protopanaxadiol-type ginsenosides Rb1 and Rb2 are known to produce stereoisomers 20(S)-Rg3 and 20(R)-Rg3 by dissociation of a glycosyl residue located at position 20 thereof by thermal processing, as illustrated in FIG. 2, followed by a dehydration reaction at position 20 to produce ginsenosides Rg5 and Rk1 (see Kang et al., Biol. Pharm. Bull., 30(4), pp. 724-728, 2007 and Lee et al., Bioorg. Med. Chem. Lett., 18(16), pp. 4515-4520, 2008).

It is also known that in such a processing process, saponins predominantly contained in Panax species plants including ginseng, i.e., ginsenosides Rb1, Rb2, Rc, and Rd are converted into ginsenosides Rg3, Rg5, and Rk1 that are not originally present in ginseng and also a variety of new pharmaceutically effective ingredients are produced, whereby antioxidative effects, anticancer effects, and blood circulation-improving effects are significantly improved (see Kim W Y et al., J. Nat. Prod., 63(12), pp. 1702-1704, 2000 and Kwon S W et al., J. Chromatogr A., 921(2), pp. 335-339, 2001).

Korean Patent No.: 0337471 discloses that an extract of a processed ginseng obtained by heating ginseng at a temperature of 110 to 180° C. for 0.5 to 20 hours contains ginsenosides Rg3, Rg5, and Rk1 as main ingredients, and the extract thereof has an inhibitory effect on nephrotoxicity. In addition, it is reported that ginsenosides Rk3 and Rh4 contained in a small amount in the processed ginseng extracts and aglycones of ginsenosides, such as panaxadiol (PD), panaxatriol (PT), protopanaxadiol (PPD), protopanaxatriol (PPT), dehydroprotopanaxadiol (DHPPD)-I, DHPPD-II, dehydroprotopanaxatriol (DHPPT)-I, and DHPPT -II have an effect of preventing or treating renal diseases (see Korean Patent No.: 0828192).

As described above, a variety of studies on protective activities of saponins of ginseng for renal protective activity have been conducted, but sufficiently effective ingredients have not been adequately developed. Therefore, there is still a need to develop medicaments with more effectively preventive or a therapeutic activity for renal diseases.

### SUMMARY OF THE INVENTION

Therefore, the inventors of the present invention intensively studied the development of a medicament for effectively preventiveor therapeutic activity for renal disease and found that Maillard browning reaction products obtained by reacting an extract of Panax species plant including ginseng or a particular ginsenoside with amino acid have a high antioxidative activity and an activity for protecting renal cells, thus completing the present invention.

An objective of the present invention is to provide a pharmaceutical composition for preventing or treating renal diseases which includes a Maillard browning reaction product of an extract of Panax species plant or a particular ginsenoside.

Another objective of the present invention is also to provide a health functional food composition for preventing or improving renal diseases which includes a Maillard browning reaction product of an extract of Panax species plant or a particular ginsenoside.

According to an aspect of the present invention, there is provided a pharmaceutical composition for preventing or treating renal diseases which includes as an active ingredient a Maillard browning reaction product obtained by reacting ginsenoside Re, an extract of Panax species plant including ginsenoside Re, or ginsenoside-derived saccharide with amino acid at a temperature of 100 to 130° C.

According to another aspect of the present invention, there is provided a health functional food composition for preventing or improving renal diseases which includes as an active ingredient a Maillard browning reaction product obtained by reacting ginsenoside Re, an extract of Panax species plant including ginsenoside Re, or ginsenoside-derived saccharide with amino acid at a temperature of 100 to 130° C.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates a table showing classification, names, and structures of dammarane-type saponins contained in Panax species plant, according to an embodiment;

FIG. 2 illustrates a reaction scheme for explaining a process in which chemical structures of ginsenosides Rb1 and Rb2 are changed by thermal processing, according to an embodiment;

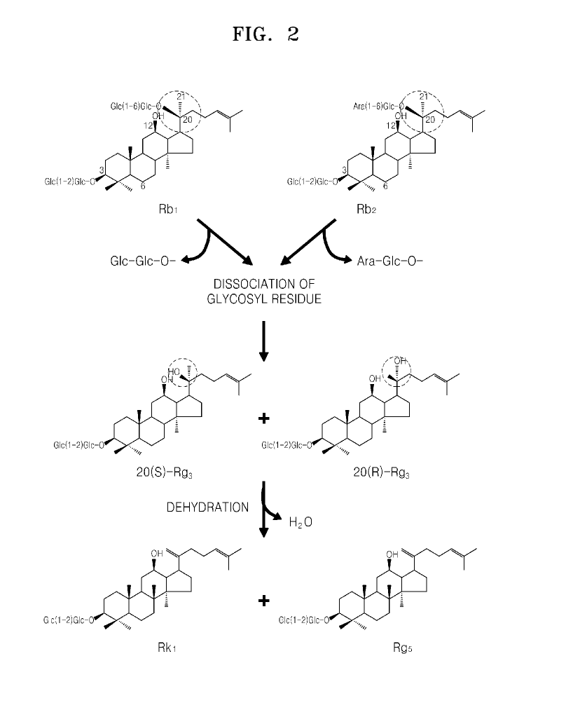

FIG. 3 illustrates a change in chemical structure of ginsenoside Re when being subjected to a Maillard browning reaction and a resultant material of a browning reaction of the separated glucose, according to an embodiment;

FIG. 4 illustrates high performance liquid chromatography (HPLC) chromatograms obtained as a result of analysis of saponin contained in each of reactants and a product of a Maillard browning reaction between ginsenoside Re and amino acid, according to an embodiment;

FIG. 5 is a graph showing measurement results of α-α-diphenyl-β-picrylhydrazyl (DPPH) radical scavenging abilities before and after a simple high-temperature reaction of ginsenoside Re at 120° C., according to an embodiment;

FIG. 6 is a graph showing measurement results of a DPPH radical scavenging ability of each of reactants and a product of a Maillard browning reaction between ginsenoside Re and glycine, according to an embodiment;

FIG. 7 is a graph showing measurement results of a DPPH radical scavenging ability of each of reactants and a product of a Maillard browning reaction between ginsenoside-derived glucose and glycine, according to an embodiment;

FIG. 8 is a graph showing measurement results of a DPPH radical scavenging ability of each of reactants and a product of a Maillard browning reaction between ginsenoside Re and leucine, according to an embodiment;

FIG. 9 is a graph showing measurement results of a DPPH radical scavenging ability of each of reactants and a product of a Maillard browning reaction between ginsenoside-derived glucose and leucine, according to an embodiment;

FIG. 10 is a graph showing measurement results of a DPPH radical scavenging ability of each of reactants and a product of a Maillard browning reaction between ginsenoside Re and serine, according to an embodiment;

FIG. 11 is a graph showing measurement results of a DPPH radical scavenging ability of each of reactants and a product of a Maillard browning reaction between ginsenoside-derived glucose and serine, according to an embodiment;

FIG. 12 is a graph showing measurement results of a DPPH radical scavenging ability of each of reactants and a product of a Maillard browning reaction between ginsenoside Re and alanine, according to an embodiment;

FIG. 13 is a graph showing measurement results of a DPPH radical scavenging ability of each of reactants and a product of a Maillard browning reaction between ginsenoside-derived glucose and alanine, according to an embodiment;

FIG. 14 is a graph showing measurement results of the amount of protein in urine after administering a Maillard browning reaction product of glucose with leucine (Control: Wild-type, Cisplatin: Cisplatin-administered group, Cisplatin+Glu-Leu: A group to which cisplatin and a Maillard browning reaction product of glucose with leucine are administered), according to an embodiment; and

FIG. 15 is a graph showing measurement results of the amount of creatinine in blood after administering a Maillard browning reaction product of glucose with leucine (Control: Wild-type, Cisplatin: Cisplatin-administered group, Cisplatin+Glu-Leu: A group to which cisplatin and a Maillard browning reaction product of glucose with leucine are administered), according to an embodiment.

### DETAILED DESCRIPTION OF THE INVENTION

Hereinafter, one or more embodiments of the present invention will be described in detail with reference to the accompanying drawings.

Unless otherwise defined, all technical terms used herein have the same meaning as commonly understood by one of ordinary skill in the art to which the present invention pertains. In addition, exemplary methods or samples are described in the present specification, but it should be understood that those similar or equivalent thereto are also encompassed in the scope of the invention. All references cited herein are hereby incorporated by reference.

The inventors of the present invention had studied ingredients of ginseng to develop an effective medicament for preventing, improving, or treating renal diseases and verified that a Maillard browning reaction product obtained during thermal processing of ginseng had a protective activity against nephrotoxicity. In particular, as a result of measuring an α-α-diphenyl-β-picrylhydrazyl (DPPH) radical scavenging activity of a Maillard browning reaction product obtained by reacting ginsenoside Re contained in an extract of Panax species plant with amino acid for a predetermined period of time, the Maillard browning reaction product exhibited a high ability to scavenge reactive oxygen species. In addition, the Maillard browning reaction product had a high activity to inhibit damage of renal epithelial cells from nephrotoxicity induced by a radical releasing reagent, i.e., 2,2′-Azobis(1-aminopropane)dihydrochloride (AAPH). Moreover, it was confirmed that a Maillard browning reaction product obtained by reacting saccharide dissociated during the thermal processing of ginsenoside with amino acid at a high temperature for a predetermined period of time also had the ability to scavenge reactive oxygen species and an activity of inhibiting the damage to renal epithelial cells, like the Maillard browning reaction product described above.

Therefore, according to one embodiment of the present invention, there is provided a pharmaceutical composition for preventing or treating a renal disease comprising as an active ingredient a Maillard browning reaction product obtained by reacting ginsenoside Re, an extract of Panax species plant including ginsenoside Re, or ginsenoside-derived saccharide with amino acid at a temperature of 100 to 130° C.

According to another embodiment of the present invention, there is provided a health functional food composition for preventing or improving a renal disease comprising as an active ingredient a Maillard browning reaction product obtained by reacting ginsenoside Re, an extract of Panax species plant including ginsenoside Re, or ginsenoside-derived saccharide with amino acid at a temperature of 100 to 130° C.

The pharmaceutical composition and the health functional food composition according to the present embodiments are also collectively referred to herein as “compositions according to the present invention.”

The Maillard browning reaction product included as an active ingredient in the compositions according to the present invention may be obtained by reacting ginsenoside Re, an extract of Panax species plant including ginsenoside Re, or ginsenoside-derived saccharide with amino acid at a temperature of 100 to 130° C. The reacting process may be performed, for example, for about 0.5 to about 12 hours, particularly, for about 2 to about 6 hours, more particularly, for about 3 to about 5 hours. The Maillard browning reaction product may be used as it is, but may be used in the form of a material that is dried at a temperature of 40 to 80° C., for example, 50 to 70° C. or freeze-dried.

The term “Maillard browning reaction product” used herein means a browned complex mixture obtained by reacting the carbonyl group of reducing sugar with the nucleophilic amino group of amino acid at a high temperature, and is well known in the art. The Maillard browning reaction product included as an active ingredient in the compositions according to the present invention may be obtained by reacting ginsenoside Re, an extract of Panax species plant including ginsenoside Re, or ginsenoside-derived saccharide with amino acid at a temperature of about 100 to about 130° C., particularly, for about 0.5 to about 12 hours.

The ginsenoside Re used as a raw material of the Maillard browning reaction product may be represented by Formula 1 below.

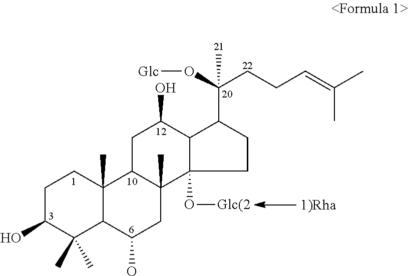

The ginsenoside Re may be obtained using a well-known method in the art, and its preparation method is not particularly limited. The ginsenoside Re may be separated from an extract of Panax species plant by using a well-known method in the art or may be obtained using a dammarane compound present in nature as a starting material by semisynthesis. For example, the ginsenoside Re may be obtained by heating Panax species plant known to contain ginsenoside Re at a temperature of about 50 to 200° C., for example, about 120° C., for 30 minutes to 10 hours, for example, for about 3 hours; adding a solvent selected from lower alcohols and mixtures thereof, for example, CH₃OH, to the heated Panax species plant, and reflux-extracting and filtering the resultant mixture at least once, for example, three times; mixing the obtained filtrates together and vacuum concentrating the filtrate mixture to obtain a CH₃OH extract; drying the CH₃OH extract under reduced pressure to remove the solvent therefrom; suspending the residue in water; separating the suspended residue into a CH₂Cl₂ fraction, a water-saturated n-BuOH fraction, and an H₂O fraction by sequentially using CH₂Cl₂ and a water-saturated n-BuOH; and performing chromatography on the water-saturated n-BuOH fraction. The water-saturated n-BuOH fraction is dried, only fractions containing ginsenoside Re are collected therefrom by column chromatography by using water, ethanol, or a mixed solvent thereof as a developing solvent, and fractions containing at least 50% of ginsenoside Re are collected therefrom and concentrated. In this regard, when the column chromatography is repeatedly performed, the amount of the ginsenoside Re may be increased. In addition, fractions containing the increased amount of the ginsenoside Re may be crystallized in an appropriate solvent system such as water, lower alcohol, lower ketone, chloroform, or a mixed solvent thereof to obtain pure ginsenoside Re.

The extract of Panax species plant including ginsenoside Re, which is used as a raw material of the Maillard browning reaction product, is not particularly limited as long as it contains ginsenoside Re. The extract of Panax species plant may be obtained from any Panax species plant including ginsenoside Re, and may be prepared using a well-known method in the art. Examples of these Panax species plants may include, but are not limited to, Panax ginseng, Panax quinquefolia, Panax notoginseng, Panax japonica, Panax trifolia, Panax pseudoginseng, Panax vietnamensis, and combinations thereof. For example, the Panax species plant may be ginseng.

The extract of Panax species plant including ginsenoside Re may be a crude extract or a product purified by additional solvent fraction or chromatography. For example, the extract of Panax species plant including ginsenoside Re may be a crude extract of water, C₁-C₄ alcohol, or a mixture thereof of any Panax species plant including ginsenoside Re; a solvent fraction of n-hexane, methylenechloride, ethylacetate, n-butanol or a mixture thereof of the crude extract; or a purified product of the solvent fraction. The crude extract of water, C₁-C₄ alcohol, or a mixture thereof may be, for example, a crude extract of methanol or ethanol, and when extracted, the amount of solvent used may be about 5 to about 15 times greater than that of Panax species plant including ginsenoside Re, for example, about 10 times, but is not limited thereto. After adding the solvent to the Panax species plant, the Panax species plant including ginsenoside Re may be extracted using one of general methods such as heating extraction, ultrasonic extraction, and reflux extraction, particularly, ultrasonic extraction, but the extraction method is not limited to the above examples. In the extraction process, the temperature of the solvent may be about 40 to about 100° C., particularly, at about 80° C., but is not limited thereto. In addition, the extraction time may be about 2 to about 4 hours, particularly, about 3 hours, but is not limited thereto. In addition, the extraction process may be performed once to five times, for example, three times, but is not limited thereto. A crude extract obtained by the above-described method may be used as the extract of Panax species plant including ginsenoside Re. Alternatively, a solvent fraction obtained by additionally extracting the crude extract with an organic solvent may be used as the extract of Panax species plant including ginsenoside Re. The solvent fraction may be a fraction obtained by extracting the crude extract with methylenechloride, hexane, ethylacetate, butanol, or a mixture thereof, but is not limited thereto.

A further purified product of the solvent fraction described above may be used as the extract of Panax species plant including ginsenoside Re. For example, ginsenoside Re may be further purified by column chromatography. The ginsenoside Re may be separated and purified by column chromatography, for example, column chromatography using a filler selected from the group consisting of silicagel, Sephadex, RP-18, polyamide, Toyopearl, and an XAD resin. The column chromatography may be performed several times by appropriately selecting a filler if desired.

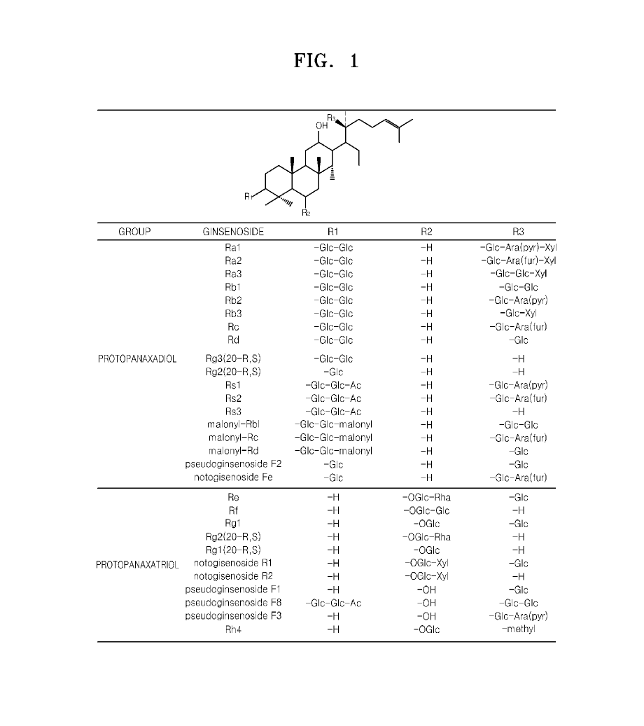

The ginsenoside-derived saccharide used as a raw material of the Maillard browning reaction product means saccharide dissociated by thermal treatment of ginsenoside contained in Panax species plant. In particular, the ginsenoside-derived saccharide may be saccharide located at R₃ of ginsenoside illustrated in FIG. 1. For example, the ginsenoside-derived saccharide may be glucose, arabinose, xylose, or a combination thereof, but is not limited thereto. Preferably, the ginsenoside-derived saccharide may be glucose dissociated from ginsenoside Re. For example, it is known that when ginsenoside Re is thermally processed at a temperature of 100 to 130° C. for at least about 0.5 hours, glucose located at position 20 is dissociated therefrom. A method of dissociating saccharide from other ginsenosides may be performed using an any methods known in the art. When a Maillard browning reaction is performed by reacting such a ginsenoside-derived saccharide with amino acid at a temperature of 100 to 130° C. for about 0.5 to about 12 hours, the Maillard browning reaction product exhibits a high antioxidative activity and a high protective activity for renal cells.

The amino acid used as a raw material of the Maillard browning reaction product may be any amino acids known to enable a Maillard browning reaction to proceed. For example, the amino acid may be glycine, alanine, cysteine, aspartic acid, glutamic acid, phenylalanine, histidine, isoleucine, lysine, leucine, methionine, asparagine, proline, glutamine, arginine, serine, threonine, selenocysteine, valine, tryptophan, tyrosine, a combination thereof, but is not limited thereto. Preferably, the amino acid may be glycine, alanine, leucine, serine, a combination thereof, but is not limited thereto. The amino acid may be used in a sufficient amount to allow the Maillard browning reaction to proceed. For example, the amount of the amino acid may be 10% to 200% of the weight of ginsenoside or glucose.

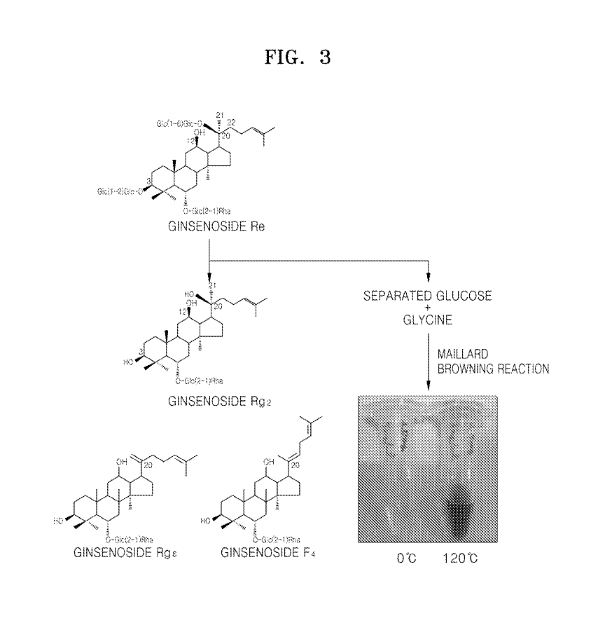

The Maillard browning reaction product included as an active ingredient in the compositions according to the present invention is confirmed to contain ginsenosides Rg2, Rg6, and F4 obtained as a result of conversion of ginsenoside Re by thermal processing (see Experimental Example 1). This is done assuming that during the Maillard browning reaction of a protopanaxatriol-based ginsenoside Re by thermal processing, a glycosyl residue located at position 20 of the ginsenoside Re is dissociated in such a manner as a reaction illustrated in FIG. 3 to thus produce ginsenoside Rg2, followed by a dehydration reaction at the 20^{th} carbon site of the ginsenoside Re to produce ginsenosides Rg6 and F4. In addition, by reacting the dissociated glucose with amino acid, a Maillard browning reaction proceeds. The conversion of ginsenoside Re by the Maillard browning reaction is illustrated in FIG. 3.

FIG. 3 illustrates a change in chemical structure of ginsenoside Re when being subjected to a Maillard browning reaction and a resultant product of a browning reaction of the dissociated glucose, according to an embodiment.

In this embodiment, the DPPH radical scavenging activity of a Maillard browning reaction product of ginsenoside Re or ginsenoside Re-derived glucose included in the compositions according to the present invention is measured, and an inhibiting effect of the Maillard browning reaction product on damage to renal epithelial cells by nephrotoxicity caused by a radical releasing agent, i.e., AAPH, is evaluated.

In the experiment for the DPPH radical scavenging activity, DPPH is a stable free radical and exhibits a peak absorbance at around 517 nm due to its non-covalent electrons and has a reduced absorbance at around 517 nm when receiving electrons or hydrogen, and thus, the antioxidative activity and scavenging activity against other radicals as well as reactive oxygen of the Maillard browning reaction product may be evaluated by measuring a decrease in absorbance (Hatano et al., Chem. Pharm. Bull., 37(8), pp 2016-2021, 1989).

Renal cells, i.e., LLC-PK1 cell lines, are vulnerable to oxidative damage, and AAPH, which is a radical releasing agent, combines with oxygen molecules at a very rapid speed to produce a peroxyl radical and trigger lipid peroxidation, resulting in cytotoxicity (Miki et al., Arch. Biochem. Biophy., 258(2), 373-380, 1987). Thus, a protective activity for renal cells may be evaluated using such an oxidative damage of the LLC-PK1 cell lines using AAPH.

As a result of evaluation on the DPPH radical scavenging activity and the effect of inhibiting nephrotoxicity induced by AAPH, the Maillard browning reaction product of ginsenoside Re or ginsenoside Re-derived glucose exhibits a higher ability to scavenge reactive oxygen (see Experimental Example 2) and a higher activity to inhibit damage to renal epithelial cells caused by nephrotoxicity induced by AAPH (see Experimental Example 3) than those of a product obtained by simply reacting ginsenoside Re at a high temperature or a mixture of ginsenoside Re and glycine.

Therefore, it is obvious that the compositions according to the present invention have an effect of preventing, improving, or treating renal diseases of mammals including humans which occur by oxidative stress as a main cause. These renal diseases may be renal diseases known to occur by oxidative stress or cell damage thereby in the art. Examples of the renal diseases include, but are not limited to, nephritis, pyelitis, nephrotic syndrome, renal cancer, acute pyelonephritis, chronic pyelonephritis, renal tuberculosis, urinary tract infection, ureterolithiasis, ureteral stone, acute renal failure, chronic renal failure, diabetic nephropathy, chronic glomerulonephritis, acute progressive nephritis, nephrotic syndrome, focal segmental glomerulosclerosis, membranous glomerulonephritis, and membranoproliferative glomerulonephritis.

A degree of inhibiting nephrotoxicity induced by an anticancer agent of the Maillard browning reaction product of ginsenoside Re-derived glucose included in the compositions according to the present invention was evaluated by measuring the amount of protein in urine and the amount of creatinine in blood. As a result of evaluation, it is confirmed that the amount of protein in urine of a group to which cisplatin is administered along with a Maillard browning reaction product of glucose and leucine is reduced by about 70% compared to that of a cisplatin-administered group, which exhibits a concentration of protein in a wild-type, and the concentration of creatinine in blood thereof is reduced by 25% compared to that of the cisplatin-administered group. According to the results, the Maillard browning reaction product of the ginsenoside Re-derived glucose may effectively improve a reduction in renal function induced by an anticancer agent, i.e., cisplatin, which means that the Maillard browning reaction product is capable of protecting renal damage induced by an anticancer agent.

Therefore, it is obvious that the compositions according to the present invention have an effect of preventing, improving, or treating renal diseases occurring in mammals including humans by administration of an anticancer agent, which are a main cause of the renal diseases. The anticancer agent may be an anti-metabolic anticancer agent including methotrexate (MTX), 5-fluorouracil (5-FU), 6-mercaptopurine (6-MP), or 6-thioguanine (6-TG); an anthracycline-based antibiotic agent such as doxorubicin or daunorubicin or an antibiotic anticancer agent including bleomycin; a microtubule inhibitor including taxol or oncovin; a steroid-based anticancer agent including prednisolone; or a platinum anticancer agent or an alkylating anticancer agent including nitrogen mustards, but is not limited thereto. According to an embodiment, the anticancer agent may be a platinum anticancer agent, and the platinum anticancer agent may be cisplatin, carboplatin, or the like, but is not limited thereto. The renal diseases may be any renal diseases known in the art to occur as side effects by anticancer agents. Examples of the renal diseases include, but are not limited to, nephritis, pyelitis, nephrotic syndrome, renal cancer, acute pyelonephritis, chronic pyelonephritis, renal tuberculosis, urinary tract infection, urolithiasis, urinary stone, urothiasis, ureterolithiasis, acute renal failure, chronic renal failure, diabetic nephropathy, chronic glomerulonephritis, acute progressive nephritis, nephrotic syndrome, focal glomerular sclerosis, membranous glomerulonephritis, and membranoproliferative glomerulonephritis.

The pharmaceutical composition according to the present invention may be formulated in the form of a general pharmaceutical formulation known in the art. The pharmaceutical formulation may include an orally-administered formulation, an injection, a suppository, a transdermally-administered formulation, and transnasally-administered formulation. However, the pharmaceutical formulations are not limited to the above examples, and may be administered in the form of any formations. Preferably, the pharmaceutical composition may be formulated in the form of formulations for oral administration, including liquid dosage forms, such as solutions, emulsions, suspensions, extracts, or syrups, and solid dosage forms, such as powders, granules, tablets, capsules, or pills.

When being formulated into each formulation, the pharmaceutical composition may be prepared by adding a pharmaceutically acceptable excipient or additive that is needed for the preparation of each formulation. For example, when formulating into a solid dosage form for oral administration, the pharmaceutically acceptable excipient may be at least one selected from a diluent, a lubricant, a binder, a disintegrant, a sweetener, a stabilizer, and a preservative. The excipient may be any excipients that are pharmaceutically acceptable. In particular, the excipient may be lactose, corn starch, soybean oil, microcrystalline cellulose, or mannitol, the lubricant may be magnesium stearate or talc, and the binder may be polyvinylpyrrolidone or hydroxypropylcellulose. In addition, the disintegrant may be calcium carboxymethyl cellulose, sodium starch glycolate, polacrilin potassium, or crospovidone. When formulating into a liquid dosage form for oral administration, various kinds of excipients, as well as a frequently used simple diluent, such as water or liquid paraffin, for example, a wetting agent, a sweetener, a flavoring agent, and a preservative, may be used. For example, the sweetener may be sucrose, fructose, sorbitol, or aspartame. The stabilizer may be sodium carboxymethyl cellulose, beta-cyclodextrin, bleached bees wax, or xanthan gum. The preservative may be methyl ρ-hydroxybenzoate, propyl ρ-hydroxybenzoate, or potassium sorbate. In addition, an additive of a solid or liquid oral formulation may be at least one selected from flavors, vitamins, and antioxidants. As a known flavoring agent in addition to the above-described ingredients, natural flavors such as Japanese apricot flavor, lemon flavor, pineapple flavor, and herb flavor; natural colors such as natural fruit juice, chlorophylin, or flavonoid; a sweetening ingredient such as fructose, honey, sugar alcohol, or sugar; or an acidifier such as citric acid or sodium citrate may be used in combination.

The general pharmaceutical formulations may be appropriately prepared by those of ordinary skilled in the art by using a general method that is well known in the art, and Remington's Pharmaceutical Science, 15th Edition, 1975, Mack Publishing Company Easton, Pa. 19042 (Chapter 87; Blaug, Seymour) may be used for reference purpose.

The pharmaceutical composition may be administered several times such that a total daily dosage of the Maillard browning reaction product as an active ingredient for preventing or treating the renal diseases is about 0.01 mg/kg to 10 g/kg, preferably, about 1 mg/kg to about 1 g/kg per adult. The dosage may be appropriately increased or decreased according to an administration route, the degree of disease progress, gender, age, body weight, or clinical diagnosis of experts. The pharmaceutical composition may be used alone or along with operation, hormone therapy, chemotherapy, and a biological response modifier therapy.

The health functional food composition may be formulated in the form of a general health functional food formulation known in the art.

The health functional food may be prepared in the form of general dosage forms such as powders, granules, tablets, pills, capsules, suspensions, emulsions, syrups, infusions, liquids and solutions, or extracts. In addition, the health functional food may be prepared in any forms of health functional foods such as meat, sausage, bread, chocolate, candies, snacks, confectionery, pizza, ramen, other noodles, gums, jelly, dairy products including ice cream, any kind of soups, beverages, tea, a health drink, alcoholic beverages, or a vitamin complex. To formulate the health functional food, a sitologically acceptable carrier or additive, and any carrier or additive known in the art that is used in the preparation of a formulation to be formulated may be used. Examples of the additive include a variety of nutritional supplements, a vitamin, an electrolyte, a flavoring agent, a coloring agent, pectic acid and salts thereof, alginic acid and salts thereof, organic acid, a protective colloidal thickener, a pH adjuster, a stabilizing agent, an antiseptic, glycerin, alcohol, and a carbonating agent used in a carbonated beverage. In addition, the additive may include a pulp for preparation of a natural fruit and vegetable juice. These additives may be used alone or in combination. A content of such an additive is inconsequential, but may be selected from a range of 0.01 to 0.1 parts by weight, based on 100 parts by weight of the composition of the invention.

In the health functional food composition, a content of the Maillard browning reaction product may be appropriately determined according to the usage purpose (prevention or improvement). In general, the content of the Maillard browning reaction product may be from 0.01 to 15 wt % based on a total weight of the health functional food composition. When the health functional food composition is prepared as a beverage, the content of the Maillard browning reaction product may be 0.02 to 10 g, preferably, 0.3 to 1 g, based on 100 ml of the health functional food composition.

The beverage may further include other ingredients as well as the active ingredient, and may further include a variety of flavoring agents or natural carbohydrates that are generally used in beverages. Non-limiting examples of the natural carbohydrates include general carbohydrates such as monosaccharides (e.g., glucose and fructose), disaccharides (e.g., maltose and sucrose), polysaccharides (e.g., dextrin and cyclodextrin) and sugar alcohols such as xylitol, sorbitol, and erythritol. In addition, examples of the flavoring agent include natural flavoring agents (e.g., thaumatin and stevia extracts) and synthetic flavoring agents (e.g., saccharin and aspartame). The content of the natural carbohydrate may be generally about 1 to about 20 g, preferably, about 5 to about 12 g, based on 100 ml of the beverage.

One or more embodiments of the present invention will now be described more fully with reference to the following examples. However, these examples are provided only for illustrative purposes and are not intended to limit the scope of the present invention.

### EXAMPLE 1

### Preparation of Ginseng Extract Including Ginsenoside Re

Ginseng used in the present invention was purchased from herbal medicine shops in the Geumsan ginseng market, Korea. 50% ethanol (1.5 L) was added to 200 g of finely cut ginseng and the resultant mixture was reflux extracted at 70° C. for 3 hours to obtain a 50% ethanol extract. Thereafter, the obtained 50% ethanol extract was dried under reduced pressure to vaporize the solvent therefrom to obtain 27 g of a dried extract including ginsenoside Re.

### EXAMPLE 2

### Separation of Ginsenoside Re

Ginsenoside Re was separated and purified from the 50% ethanol extract prepared according to Example 1 by silicagel column and semi-preparative high-performance liquid chromatography (HPLC). 20 g of a 50% ethanol extract obtained using the same extraction method as that used in Example 1 was suspended in water and stirred along with 100 g of a nonpolar DIAION HP 20 resin in a 1 L glass container. The resultant resin was washed three times with purified water and eluted with methyl alcohol to obtain a methyl alcohol fraction. The methyl alcohol fraction was dried under reduced pressure to obtain 1.9 g of a saponin fraction. The saponin fraction was subjected to silicagel column chromatography in mixed solvents of hexane: ethylacetate (10:1→5:1→1:1), and only a ginsenoside Re-containing fraction was separated, collected, and concentrated. A fraction containing at least 50% of ginsenoside Re was eluted by semi-preparative HPLC so that a ratio of water to CH₃CN as a mobile phase reached 0:100 in 40 minutes from 40:60, thereby obtaining 50 mg of pure ginsenoside Re.

### EXAMPLE 3

### Preparation of Maillard Browning Reaction Product of Ginsenoside Re or Ginsenoside Re-Derived Saccharide with Amino Acid

The ginsenoside Re prepared according to Example 2 or ginsenoside Re-derived glucose were thermally processed with amino acid using the method disclosed in the publication (Lee et al., Bioorg. Med. Chem. Lett., 18(16), 4515-4520, 2008). According to the above-described publication, it was confirmed that ginsenoside Re-derived glucose had the same chemical structure as that of glucose commercially available as a reagent, and thus, ginsenoside Re-derived glucose purchased from Sigma-Aldrich Korea Ltd., was used.

In particular, 20 mg of the ginsenoside Re or 48 mg of glucose were heated in an autoclave along with 20 mg of glycine, 20 mg of leucine, 20 mg of serine, or 20 mg of alanine at 120° C. for 3 hours and the resultant mixture was freeze-dried to obtain a Maillard browning reaction product. A change in chemical structure of the ginsenoside Re when being subjected to a Maillard browning reaction and a resultant product of the Maillard browning reaction of the separated glucose are shown in FIG. 3.

From the results, it was confirmed that glucose located at the 20^{th} carbon site of ginsenoside Re which was dissociated by thermal processing of the ginsenoside Re triggered a Maillard browning reaction together with glycine, leucine, serine, or alanine to produce a Maillard browning reaction product as shown in FIG. 3.

### COMPARATIVE EXAMPLE 1

### Preparation of Simple High-Temperature Treatment Product of Ginsenoside Re

Ginsenoside Re was high-temperature treated at 120° C. in the same manner as in Example 3 to obtain a product, except that amino acid was not added to the ginsenoside Re.

### EXPERIMENTAL EXAMPLE 1

### Analysis of Maillard Browning Reaction Product

1-1. Experimental Method

Saponins before and after a Maillard browning reaction between the ginsenoside Re prepared according to Example 3 and glycine were analyzed by HPLC disclosed in the publication (Kwon et al., J. Chromatogr. A, 921(2), 335-339, 2001), by using as a control a standard solution containing each of ginsenosides Re, Rg2, Rg6, and F4.

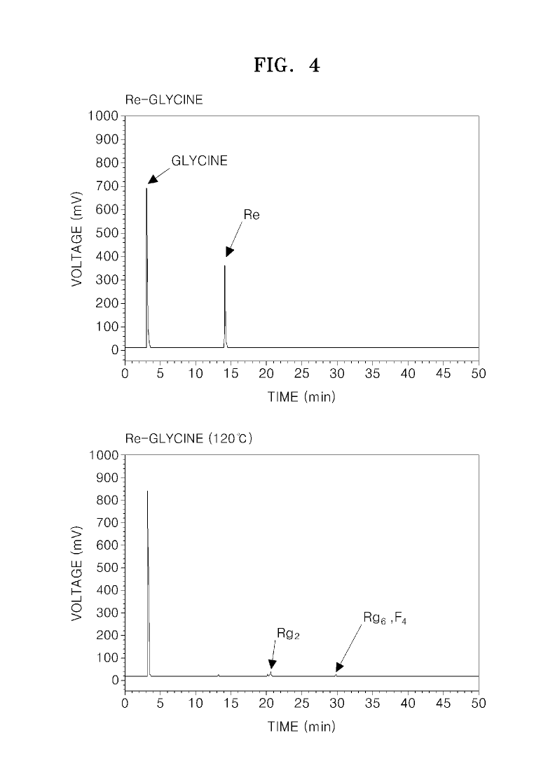

Eluting solvents, i.e., water (H₂O) and acetonitrile (CH₃CN), were each filtered through a 0.45 μm membrane filter and were respectively used by pumping with two pumps. 10 μl of the standard solution was injected into a separation column, i.e., a reverse phase column (C18 column, 4.6×150 mm, 5 μm) by using a syringe, and the eluting solvent containing 30 volume% of acetonitrile and 70 volume % of water was made to flow through the reverse phase column at a flow rate of 1 ml/min. Thereafter, the content of acetonitrile was made so as to reach 100 volume % within 40 minutes, and the content of acetonitrile (i.e., 100 volume %) was maintained for 15 minutes. After the foregoing process, a peak of each constituent separated from the separation column was obtained using an evaporative light scattering detector (ELSD), and the results are shown in FIG. 4.

FIG. 4 illustrates HPLC chromatograms obtained as a result of analysis of saponins before and after a Maillard browning reaction of ginsenoside Re with glycine. In addition, saponins before and after a Maillard browning reaction of ginsenoside Re with leucine, serine, or alanine were analyzed. As a result of analysis, a peak having a similar pattern to that shown in FIG. 4 was observed.

1-2. Experimental Results

From the results shown in FIG. 4, it was shown that ginsenoside Re was thermally processed at 120° C. along with amino acid, thereby being transformed into ginsenoside Rg2, Rg6, or F4 by dissociation of glucose located at position 20 of ginsenoside Re. Thus, it was confirmed that the ginsenoside Re underwent a change in chemical structure as illustrated in FIG. 3.

### EXPERIMENTAL EXAMPLE 2

### Antioxidative Activity Evaluation

2-1. Experimental Method

A DPPH radical scavenging ability was evaluated using a modified method from that described by Hatano et al (Hatano et al., Chem. Pharm. Bull., 37(8), pp 2016-2021, 1989). 100 μl of 60 μM DPPH was added to 100 μl of each of a plurality of sample solutions, the resultant solution was stirred and left for 30 minutes, and the absorbance of the resultant mixture was measured at 540 nm. An antioxidative activity (electron donating ability) was confirmed by measuring a decreasing ratio in absorbances of experimental groups comparing with a control of the sample solutions.

2-2. Experimental Results

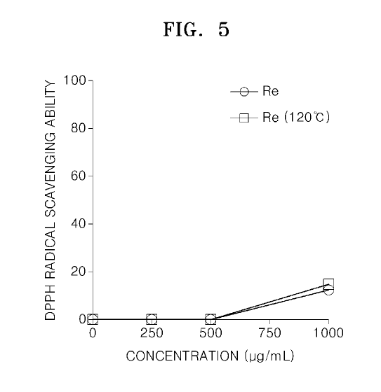

DPPH radical scavenging activities of the simple high-temperature treatment product of ginsenoside Re prepared according to Comparative Example 1 and products before and after a Maillard browning reaction between the ginsenoside Re and glycine in Example 3 were measured. The results are shown in FIGS. 5 through 13.

FIG. 5 is a graph showing measurement results of DPPH radical scavenging abilities before and after a simple high-temperature reaction of ginsenoside Re at 120° C.

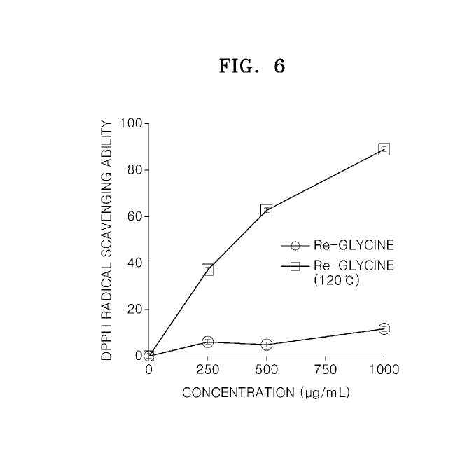

FIG. 6 is a graph showing measurement results of a DPPH radical scavenging ability of each of reactants and a product of Maillard browning reaction between ginsenoside Re and glycine according to an embodiment of the present invention.

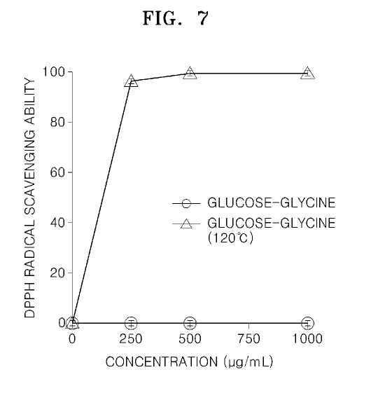

FIG. 7 is a graph showing measurement results of a DPPH radical scavenging ability of each of reactants and a product of Maillard browning reaction between ginsenoside-derived glucose and glycine according to an embodiment of the present invention.

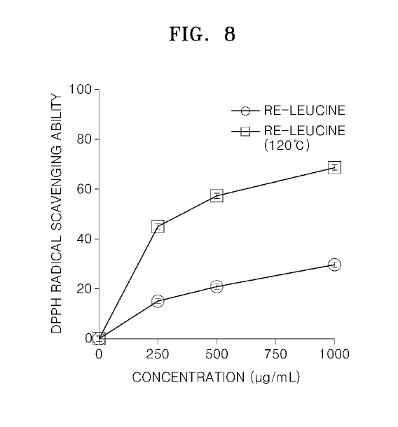

FIG. 8 is a graph showing measurement results of a DPPH radical scavenging ability of each of reactants and a product of Maillard browning reaction between ginsenoside Re and leucine according to an embodiment of the present invention.

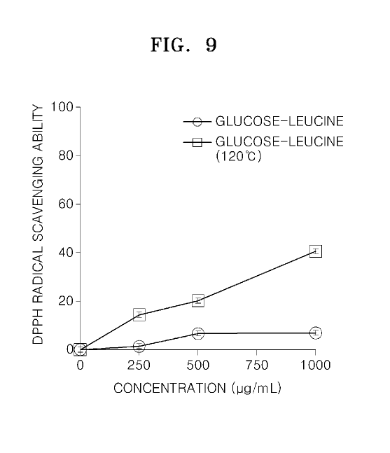

FIG. 9 is a graph showing measurement results of a DPPH radical scavenging ability of each of reactants and a product of Maillard browning reaction between ginsenoside-derived glucose and leucine according to an embodiment of the present invention.

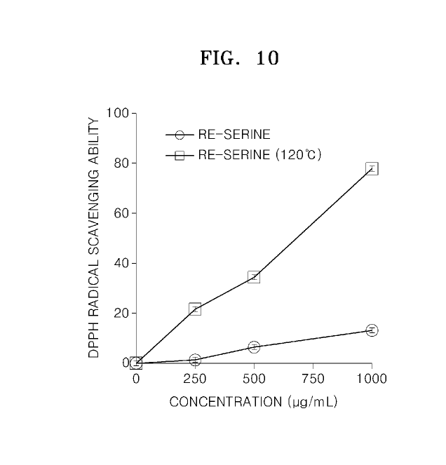

FIG. 10 is a graph showing measurement results of a DPPH radical scavenging ability of each of reactants and a product of Maillard browning reaction between ginsenoside Re and serine according to an embodiment of the present invention.

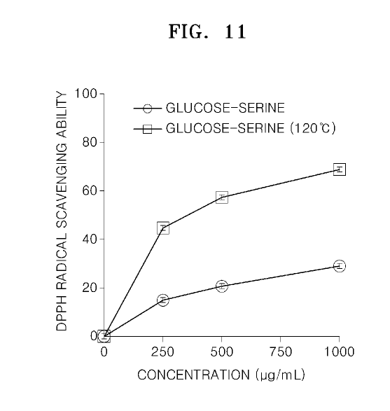

FIG. 11 is a graph showing measurement results of a DPPH radical scavenging ability of each of reactants and a product of Maillard browning reaction between ginsenoside-derived glucose and serine according to an embodiment of the present invention.

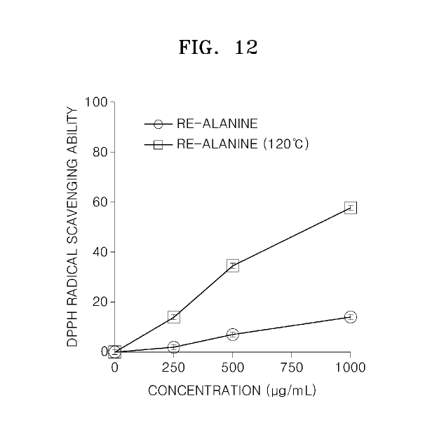

FIG. 12 is a graph showing measurement results of a DPPH radical scavenging ability of each of reactants and a product of Maillard browning reaction between ginsenoside Re and alanine according to an embodiment of the present invention.

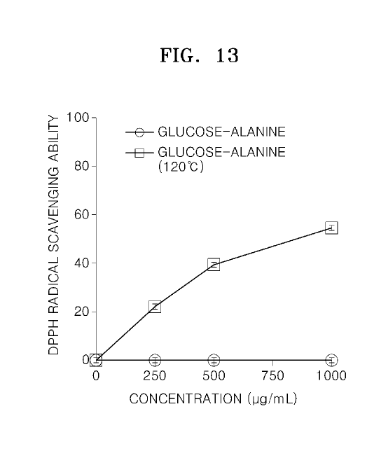

FIG. 13 is a graph showing measurement results of a DPPH radical scavenging ability of each of reactants and a product of Maillard browning reaction between ginsenoside-derived glucose and alanine according to an embodiment of the present invention.

As illustrated in FIGS. 5, 6 and 7, ginsenoside Re and a product obtained by performing simple thermal processing on ginsenoside Re at 120° C. exhibited a low reactive oxygen scavenging ability. In contrast, the Maillard browning reaction product of ginsenoside Re with glycine exhibited a higher concentration-dependent DPPH radical scavenging ability than that of a simple mixture of ginsenoside Re and glycine before the Maillard browning reaction therebetween. In addition, the Maillard browning reaction product obtained by thermally processing glycine and glucose dissociated from ginsenoside Re by thermal processing thereof at 120° C. exhibited a higher concentration-dependent DPPH radical scavenging ability than that of a simple high-temperature treatment product of ginsenoside Re.

As illustrated in FIGS. 5, 8, and 9, the Maillard browning reaction product of ginsenoside Re with leucine exhibited a higher concentration-dependent DPPH radical scavenging ability than that of a simple mixture of ginsenoside Re and leucine before the Maillard browning reaction therebetween. In addition, the Maillard browning reaction product obtained by thermally processing leucine and glucose dissociated from ginsenoside Re by thermal processing thereof at 120° C. exhibited a higher concentration-dependent DPPH radical scavenging ability than that of a simple high-temperature treatment product of ginsenoside Re.

As illustrated in FIGS. 5, 10, and 11, the Maillard browning reaction product of ginsenoside Re with serine exhibited a higher concentration-dependent DPPH radical scavenging ability than that of a simple mixture of ginsenoside Re and serine before the Maillard browning reaction therebetween. In addition, the Maillard browning reaction product obtained by thermally processing serine and glucose dissociated from ginsenoside Re by thermal processing thereof at 120° C. exhibited a higher concentration-dependent DPPH radical scavenging ability than that of a simple high-temperature treatment product of ginsenoside Re. As illustrated in FIGS. 5, 12, and 13, the Maillard browning reaction product of ginsenoside Re with alanine exhibited a higher concentration-dependent DPPH radical scavenging ability than that of a simple mixture of ginsenoside Re and alanine before the Maillard browning reaction therebetween. In addition, the Maillard browning reaction product obtained by thermally processing alanine and glucose dissociated from ginsenoside Re by thermal processing thereof at 120° C. exhibited a higher concentration-dependent DPPH radical scavenging ability than that of a simple high-temperature treatment product of ginsenoside Re.

Consequently, it was confirmed that the compositions according to the present invention had high electron donating abilities.

### EXPERIMENTAL EXAMPLE 3

### Evaluation of Protective Activity for Renal Cells

3-1. Experimental Method

A protective effect for nephrotoxicity was evaluated using renal cells (LLC-PK1) with reference to a method reported in the publication (Yokozawa et al., Food Chem., 48, pp5068-5073, 2000) as follows.

First, the LLC-PK1 cells were cultured in an incubator with conditions of 37° C., 95% air, and 5% CO₂ by using a DMEM/F12 medium (Gibco BRL Life Technologies, Grand Island, NY, USA) supplemented with 5% fetal bovine serum (Gibco BRL Life Technologies), 50 units/ml of penicillin G, and 50 μg/ml of streptomycin (Gibco BRL Life Technologies). The incubated LLC-PK1 cells were introduced into a 96-well incubation plate at a concentration of 10⁴ cells/pi and then stabilized for 2 hours. Afterwards, a radical generating reagent (i.e., 10 mM AAPH dissolved in a medium) was added to each well and cultured for 24 hours, and 50 μl of an MTT (1 mg/ml) reagent was then added to each well and incubated at 37° C. 4 hours thereafter, the medium containing MTT was removed and 100 μl of dimethyl sulfoxide was added to each well. Then, absorbance of the sample was measured using a detection wavelength of 540 nm in a SPECTRAmax 340PC (Molecular Devices, Sunnyvale, Calif., USA) microplate reader and a viability of the LLC-PK1 cells was measured using the absorbance thereof.

3-2. Experimental Result

Results of experiments using as samples the ginsenoside Re prepared according to Example 2, the simple high-temperature treatment product of ginsenoside Re prepared according to Comparative Example 1, reactants and the Maillard browning reaction product of ginsenoside Re with glycine of Example 3, the Maillard browning reaction product of glucose and glycine of Example 3, and aminoguanidine as a positive control are shown in Table 1 below.

As seen from experimental results shown in Table 1, the number of LLC-PK1 cells was reduced by 71.7% of that of the AAPH-non-treatment groups by the treatment of 10 mM AAPH. It was confirmed that Ginsenoside Re inhibited cell damage at a high concentration and ginsenoside Re that had been thermally processed at 120° C. exhibited a much higher protective activity for renal cells. In particular, while the simple mixture of ginsenoside Re and glycine had a very low protective activity for renal cells, the Maillard browning reaction product obtained by the reaction of ginsenoside Re with glycine at 120° C. increased the number of LLC-PK1 cells reduced by AAPH by 97.6% at a concentration of 10 μg/ml. In addition, the Maillard browning reaction product obtained by heat-treating at 120° C. glycine and glucose dissociated from ginsenoside Re by heat treatment meaningfully recovered damage to renal cells caused by AAPH by at least 100% at a concentration of 10 μg/ml. These effects are far much higher than those of aminoguanidine, which is a therapeutic agent for diabetic nephropathy.

TABLE 1

| | Concentration | Cell viability |
|---|---|---|
| Sample | (μg/ml) | (%) |
| ginsenoside Re | 0 | 71.7 ± 4.1 |
| | 1 | 74.5 ± .8.8 |
| | 10 | 89.6 ± 9.7^{a} |
| ginsenoside Re | 0 | 71.7 ± 4.1 |
| (120° C.) | 1 | 88.9 ± 0.9^{a} |
| | 10 | 93.7 ± 4.3^{a} |
| ginsenoside | 0 | 71.7 ± 4.1 |
| Re + glycine | 1 | 86.1 ± 3.9 |
| | 10 | 74.1 ± 6.0 |
| ginsenoside | 0 | 70.5 ± 6.6 |
| Re + glycine | 1 | 72.3 ± 7.0 |
| (120° C.) | 10 | 97.6 ± 6.4^{a} |
| glucose + | 0 | 71.5 ± 0.4 |
| glycine | 1 | 79.9 ± 3.8^{a} |
| (120° C.) | 10 | 103 ± 0.2^{a} |
| aminoguanidine | 0 | 71.7.1 |
| | 1 | 74.0 ± 3.0 ± 4 |
| | 10 | 86.7 ± 14.2 |
| | 100 | 88.0 ± 4.1^{a} |

* Statistical significance: ^{a}P < 0.05 vs. AAPH treatment standard value

Results of experiment using reactants and the Maillard browning reaction product of ginsenoside Re with leucine of Example 3, the Maillard browning reaction product of glucose with leucine of Example 3, reactants and a product of the Maillard browning reaction between ginsenoside Re and serine of Example 3, the Maillard browning reaction product of glucose with serine of Example 3, reactants and a product of the Maillard browning reaction between ginsenoside Re and alanine of Example 3, and the Maillard browning reaction product of glucose with alanine of Example 3 are shown in Table 2 below.

As seen from the experimental results shown in Table 2, the number of

LLC-PK1 cells was reduced by 77.4% of that of the AAPH-non-treatment groups by the treatment of 10 mM AAPH. In addition, while the simple mixture of ginsenoside Re and leucine had a very low protective activity for renal cells, the Maillard browning reaction product obtained by the reaction of ginsenoside Re and leucine at 120° C. increased the number of LLC-PK1 cells reduced by AAPH by 96.3% at a concentration of 10 μg/ml. Also, the Maillard browning reaction product obtained by heat-treating at 120° C. leucine and glucose dissociated from ginsenoside Re by heat treatment meaningfully recovered damage to renal cells caused by AAPH by at least 90% at a concentration of 10 μg/ml. Next, while the simple mixture of ginsenoside Re and serine had a very low renal protective activity, the Maillard browning reaction product obtained by the reaction of ginsenoside Re and serine at 120° C. increased the number of LLC-PK1 cells reduced by AAPH by 93.6% at a concentration of 10 pg/ml. Also, the Maillard browning reaction product obtained by heat-treating at 120° C. serine and glucose dissociated from ginsenoside Re by heat treatment meaningfully recovered damage to renal cells caused by AAPH by at least 90% at a concentration of 10 μg/ml. Next, while the simple mixture of ginsenoside Re and alanine had a very low renal protective activity, the Maillard browning reaction product obtained by the reaction of ginsenoside Re and alanine at 120° C. increased the number of LLC-PK1 cells reduced by AAPH by 93.1% at a concentration of 10 μg/ml. In addition, the Maillard browning reaction product obtained by heat-treating at 120° C. alanine and glucose dissociated from ginsenoside Re by heat treatment meaningfully recovered damage to renal cells caused by AAPH by at least 99.2% at a concentration of 10 μg/ml.

TABLE 2

| | Concentration | Cell viability |
|---|---|---|
| Sample | (μg/ml) | (%) |
| ginsenoside Re + leucine | 0 | 77.4 ± 2.4 |
| | 1 | 86.4 ± 1.6 |
| | 10 | 86.8 ± 4.0^{a} |
| ginsenoside Re + leucine | 0 | 77.4 ± 2.4 |
| (120° C.) | 1 | 98.3 ± 7.8^{a} |
| | 10 | 96.3 ± 1.2^{a} |
| glucose + leucine | 0 | 77.4 ± 2.4 |
| (120° C.) | 1 | 82.6 ± 3.8 |
| | 10 | 91.3 ± 3.7^{a} |
| ginsenoside Re + serine | 0 | 79.0 ± 1.6 |
| | 1 | 75.6 ± 2.9 |
| | 10 | 85.4 ± 2.0^{a} |
| ginsenoside Re + serine | 0 | 81.5 ± 1.5 |
| (120° C.) | 1 | 86.1 ± 5.4 |
| | 10 | 93.6 ± 5.8^{a} |
| glucose + serine | 0 | 81.5 ± 1.5 |
| (120° C.) | 1 | 85.4 ± 4.2 |
| | 10 | 93.5 ± 0.1^{a} |
| ginsenoside Re + alanine | 0 | 81.5 ± 1.5 |
| | 1 | 82.7 ± 3.1 |
| | 10 | 81.9 ± 7.0 |
| ginsenoside Re + alanine | 0 | 76.7 ± 6.0 |
| (120° C.) | 1 | 90.9 ± 4.5^{a} |
| | 10 | 93.1 ± 4.3^{a} |
| glucose + alanine | 0 | 75.4 ± 1.1 |
| (120° C.) | 1 | 99.2 ± 5.1^{a} |
| | 10 | 99.2 ± 3.6^{a} |

* Statistical significance: ^{a}P < 0.05 vs. AAPH treatment standard value

From the results shown in Table 1, it was confirmed that the Maillard browning reaction product obtained by heat treatment of ginsenoside Re withglycine, leucine, serine, or alanine at 120° C. and the Maillard browning reaction product obtained by heat treatment of glucose with glycine, leucine, serine, or alanine at 120° C. more effectively protected damage to renal cells by AAPH than ginsenoside Re. These results mean that the Maillard browning reaction product of dammarane-based ginsenoside Re or glucose with amino acid is capable of protecting damage to renal cells caused by oxidative stress.

### Experimental Example 4

### Evaluation of Protective effect on Nephrotoxicity Induced by Anticancer Agent

4-1. Experimental Method

A protective effect on nephrotoxicity of cisplatin, which is an anticancer agent, was evaluated as follows by using Sprague-Dawley male mice with reference to a method reported in the following document (Sahu et al., Food Chem. Toxicol., 49, pp 3090-3097, 2011).

As a laboratory animal, Sprague-Dawley mice with a body weight of 170 to 190 g were used. An overall experiment process was performed in accordance with guidelines for ethical regulation on the use of laboratory animals of the Korea

Institute of Science and Technology. To trigger cisplatin nephrotoxicity, 7.5 mg/kg of cisplatin was administered once via intraperitoneal injection. The Sprague-Dawley male mice were administered with the Maillard browning reaction product of glucose with leucine of Example 3 mixed at a concentration of 0.5 wt % with drinking water for 10 days total, i.e., 6 days before the cisplatin injection to 4 days thereafter. After the administration of the Maillard browning reaction product for 10 days, to measure renal function, the male mice were put in a metabolic cage, urines thereof were collected for 24 hours, and the amount of protein in urine was measured. In addition, the male mice were cut the abdomen open under anesthesia, blood thereof was collected, and the amount of serum creatinine was measured.

Protein in urine was measured by colorimetry using a COMBOSTIK-2GP urine test paper, and the concentration of serum creatinine was measured by Rate blank Jaffe Kinetic method using a creatinine reagent (Roche, USA) by using a Hitachi modular device (Japan).

4-2. Experimental Results

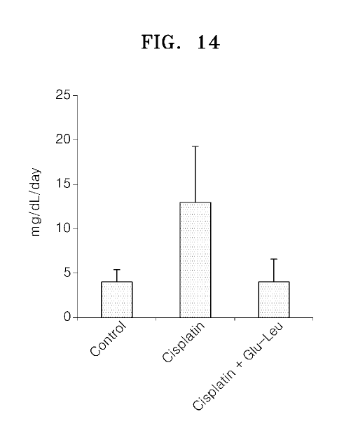

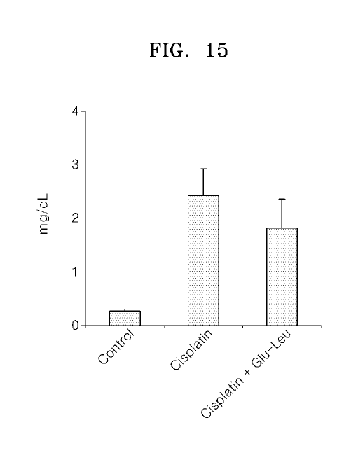

Results of experiments using as a sample the Maillard browning reaction product of glucose with leucine of Example 3 are shown in FIGS. 14 and 15.

FIG. 14 is a graph showing measurement results of the amount of protein in urine after administering a Maillard browning reaction product of glucose with leucine.

FIG. 15 is a graph showing measurement results of the amount of creatinine in blood after administering a Maillard browning reaction product of glucose with leucine.

As shown in FIGS. 14 and 15, it was confirmed that the amount of protein in urine of a group to which the Maillard browning reaction product of glucose and leucine was administered with cisplatin was reduced by about 70% as compared to a cisplatin-administered group, which corresponds to the concentration range of protein in wild-type. In addition, it was confirmed that the concentration of creatinine in blood was reduced by 25% as compared to the cisplatin-administered group.

From the results, it is confirmed that the Maillard browning reaction product of glucose with leucine effectively improves a decrease in renal function induced by cisplatin, an anticancer agent, which means that the Maillard browning reaction product is capable of protecting renal damage induced by an anticancer agent.

Preparation Examples of the compositions according to the present invention are described herein. In these examples, a Maillard browning reaction product of amino acid and ginsenoside Re or saccharide dissociated from ginsenoside Re of Example 3 was used as an active ingredient.

### PREPARATION EXAMPLE 1

### Preparation of Pharmaceutical Formulation

1. Preparation of Powders

| | |
|---|---|
| Active ingredient | 2 g |
| Lactose | 1 g |

powders were prepared by mixing the above ingredients and filling a sealed package with the resultant mixture.

2. Preparation of Tablet

| | |
|---|---|
| Active ingredient | 100 mg |
| Corn starch | 100 mg |
| Lactose | 100 mg |
| Magnesium stearate | 2 mg |

Tablets were prepared by mixing the above ingredients and tabletting the resultant mixture using a convenetional method of preparing a tablet.

3. Preparation of Capsule

| | |
|---|---|
| Active ingredient | 100 mg |
| Corn starch | 100 mg |
| Lactose | 100 mg |
| Magnesium stearate | 2 mg |

Capsules were prepared by mixing the above ingredients and filling a gelatin capsule with the resultant mixture using a conventional method of preparing capsules.

4. Preparation of Pill

| | |
|---|---|
| Active ingredient | 1 g |
| Lactose | 1.5 g |
| Glycerin | 1 g |
| Xylitol | 0.5 g |

Pills were prepared by mixing the above ingredient so that the pills contained 4 g of the ingredients per pill by using a conventional method.

5. Preparation of Granule

| | |
|---|---|
| Active ingredient | 150 mg |
| Soybean extract | 50 mg |
| Glucose | 200 mg |
| Starch | 600 mg |

The above ingredients were mixed together, 100 mg of 30% ethanol was added to the mixed ingredients, the resultant mixture was dried at 60° C. to form granules, and a packet was filled with the granules.

### PREPARATION EXAMPLE 2

### Preparation of Health Food

1. Preparation of Tomato Ketchup and Sauce

0.2 to 1.0 parts by weight of the active ingredient was added to tomato ketchup and sauce to prepare tomato ketchup or sauce for health improvement.

2. Preparation of Flour Food

0.5 to 5.0 parts by weight of the active ingredient was added to flour, and bread, cake, cookies, cracker, and noodles were prepared using the resultant mixture, thereby completing the preparation of foods for health improvement.

3. Preparation of Soup and Gravies

0.1 to 5.0 parts by weight of the active ingredient was added to soup and gravies to prepare soup and gravies of processed meat products and noodles for health improvement.

4. Preparation of Ground Beef

10 parts by weight of the active ingredient was added to ground beef to prepare ground beef for health improvement.

5. Preparation of Dairy Products

5 to 10 parts by weight of the active ingredient was added to milk and a variety of dairy products such as butter and ice cream were prepared using the resultant milk.

6. Preparation of Natural Food Products

Brown rice, barley, glutinous rice, and adlay were pregelatinized using a known method and dried, and the dried resultant mixture was roasted and pulverized using a pulverizer to prepare powders having a particle diameter of 60 mesh. Separately, black bean, black sesame, and perilla seeds were steamed using a known method and dried, and the dried resultant mixture was roasted and pulverized using a pulverizer to prepare powder having a particle diameter of 60 mesh. The active ingredient was decompression concentrated in a vacuum concentrator and dried using a spray convection dryer to obtain a dried material. Thereafter, the dried material was pulverized using a pulverizer to obtain a dried powder having a particle diameter of 60 mesh.

The prepared grains, seeds and nuts, and the dried powder of the extracts of Examples 1 and 2 were mixed at a mixing ratio as described below to prepare a final product:

Grains (30 parts by weight of brown rice, 15 parts by weight of adlay, and 20 parts by weight of barley),

Seeds and nuts (7 parts by weight of perilla seeds, 8 parts by weight of black bean, and 7 parts by weight of black sesame seeds),

3 parts by weight of the dried powder of a compound separated from the extracts of Examples 1 and 2,

0.5 parts by weight of Lingzhi mushroom

0.5 parts by weight of geogen

### PREPARATION EXAMPLE 3

### Preparation of Health Beverage

1. Preparation of Healthy Drink

| | | |
|---|---|---|
| Active ingredient | 1000 | mg |
| Citric acid | 1000 | mg |
| Oligosaccharide | 100 | g |
| Plum concentrated extract | 2 | g |
| Taurine | 1 | g |
| A total volume with addition of purified water | 900 | ml |

According to a conventional preparation method for health functional beverages, all the ingredients were blended together and heated under agitation at 85° C. The prepared solution was filtered and poured into a sterile 2 L container, which was then seal-sterilized and kept in a refrigerator. The resultant product was used to prepare a health functional beverage composition according to the present invention.

The composition as described herein was prepared by mixing ingredients relatively appropriate for beverages at a mixing ratio according to a preferred example, but the mixing ratio of the ingredients may be modified according to geographic and ethnic preferences, such as target customers, target country, usage, and the like.

2. Preparation of Vegetable Juice

5 g of the active ingredient was added to 1,000 ml of tomato or carrot juice to prepare vegetable juice for health improvement.

3. Preparation of Fruit Juice

1 g of the active ingredient was added to 1,000 ml of apple or grape juice to prepare fruit juice for health improvement.

As described above, according to the one or more embodiments of the present invention, a Maillard browning reaction product of amino acid with ginsenoside Re, an extract of Panax species plant including ginsenoside Re, or glucose has a high reactive oxygen scavenging ability and a high inhibitory activity of damage to renal epithelial cells caused by oxidative stress. Thus, the Maillard browning reaction product thereof may be effectively used for the prevention, improvement, or treatment of renal diseases. In addition, the compositions according to the present invention include ingredients of plant herbal medicine that has been long used, and thus, the stability thereof has been established. Therefore, the compositions according to the present invention may be used in the long term without concerns of side effects.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of preventing, improving, or treating a renal disease, the method comprising administering as an active ingredient a Maillard browning reaction product obtained by reacting ginsenoside Re, an extract of Panax species plant comprising ginsenoside Re, or ginsenoside-derived saccharide with amino acid at a temperature of 100 to 130° C.

2. The method of claim 1, wherein the Panax species plant is Panax ginseng, Panax quinquefolia, Panax notoginseng, Panax japonica, Panax trifolia, Panax pseudoginseng, Panax vietnamensis, a cultured root thereof, or a combination thereof.

3. The method of claim 1, wherein the extract of Panax species plant comprises a crude extract of water, C₁-C₄ alcohol, or a mixture thereof of Panax species plant; a solvent fraction of n-hexane, methylenechloride, ethylacetate, butanol, or a mixture thereof of the crude extract; or a purified material of the solvent fraction.

4. The method of claim 1, wherein the amino acid is glycine, alanine, cysteine, aspartic acid, glutamic acid, phenylalanine, histidine, isoleucine, lysine, leucine, methionine, asparagine, proline, glutamine, arginine, serine, threonine, selenocysteine, valine, tryptophan, tyrosine, or a combination thereof.

5. The method of claim 4, wherein the amino acid is glycine, alanine, leucine, serine, or a combination thereof.

6. The method of claim 1, wherein the ginsenoside-derived saccharide is glucose, arabinose, xylose, or a combination thereof.

7. The method of claim 1, wherein the renal disease is nephritis, pyelitis, nephrotic syndrome, renal cancer, acute pyelonephritis, chronic pyelonephritis, renal tuberculosis, urinary tract infection, urolithiasis, ureterolithiasis, acute renal failure, chronic renal failure, diabetic nephropathy, chronic glomerulonephritis, acute progressive nephritis, nephrotic syndrome, focal glomerular sclerosis, membranous glomerulonephritis, or membranoproliferative glomerulonephritis.

8. The method of claim 1, wherein the renal disease is a renal disease induced by an anticancer agent.

9. The method of claim 8, wherein the anticancer agent is a platinum anticancer agent.

10. The method of claim 1, wherein the Maillard browning reaction product is prepared in the form of powders, granules, tablet, capsule, suspension, emulsion, syrup, solution, aerosol, extract, injection, transdermal therapeutic system, tea, jelly, beverage, or suppository.
